(19) United States
(12) Patent Application Publication
(10) Pub. No.: US 2016/0331347 A1
(43) Pub. Date: Nov. 17, 2016

(54) OBJECT INFORMATION ACQUIRING APPARATUS

(71) Applicant: CANON KABUSHIKI KAISHA, Tokyo (JP)

(72) Inventors: Hisafumi Ebisawa, Tokyo (JP); Kenichi Nagae, Yokohama-shi (JP)

(21) Appl. No.: 15/140,611

(22) Filed: Apr. 28, 2016

(30) Foreign Application Priority Data
May 13, 2015 (JP) 2015-098270

(51) Int. Cl. A61B 8/08; A61B 5/00; A61B 8/00

(52) U.S. Cl. CPC A61B 8/5207; A61B 8/0825; A61B 8/54; A61B 8/4461; A61B 5/0077; A61B 8/406; A61B 8/461; A61B 8/5269; A61B 8/483

(57) ABSTRACT

An object information acquiring apparatus is used which includes a receiver including a plurality of elements each transmitting an acoustic wave, receiving an echo wave resulting from reflection of the acoustic wave by an object, and outputting an electric signal, a transmission controller that controls an intensity of the acoustic wave transmitted from each of the plurality of elements, a scanner that moves the receiver in a predetermined scanning region, and an information processor that acquires characteristics information on an inside of the object using the electric signal. The transmission controller controls the intensity of the acoustic wave in accordance with a shape of the object and a position of the receiver in the predetermined scanning region.

### BACKGROUND OF THE INVENTION

1. Field of the Invention

The present invention relates to an object information acquiring apparatus.

2. Description of the Related Art

With the purpose of obtaining characteristics information on the interior of an object such as the breasts, research has been ongoing on object information acquiring apparatuses that use ultrasonic waves. To mention some examples, ultrasonic apparatuses are available which irradiate the object with ultrasonic waves and receive an echo signal reflected by the object so as to generate the characteristics information, or photoacoustic apparatuses are available which irradiate the object with laser light and receive an ultrasonic wave (photoacoustic wave) that results from a photoacoustic effect so as to generate the characteristics information.

In the ultrasonic apparatus in Japanese Patent Application Laid-open No. 2008-073305, a probe arranged on a floor portion of a tank obtains three-dimensional image data by transmitting and receiving an ultrasonic wave to and from the breasts suspended and immersed in the water tank while mechanically moving in a horizontal plane. The resultant image data can be displayed on a monitor, for example, as any sectional images of the breasts.

Patent Literature 1: Japanese Patent Application Laid-open No. 2008-073305

### SUMMARY OF THE INVENTION

Hereinbelow, the direction in which the probe transmits the ultrasonic wave will be referred to as a “depth”. The probe in Japanese Patent Application Laid-open No. 2008-073305 performs scanning in the horizontal plane, and thus, a distance from the probe to a surface of each of the breasts varies between a case where the probe lies opposite to a tip portion (central portion) of the breast and a case where the probe lies opposite to a peripheral portion of the breast. Therefore, the ratio between water and the body tissue in a path of the ultrasonic wave varies between the tip portion and the peripheral portion of the breast. Moreover, the body tissue is, in general, more likely to attenuate the ultrasonic wave than water.

Thus, under the same measurement conditions, the ultrasonic wave traveling from the probe to a depth L has a lower intensity when the probe lies opposite to the tip portion of the breast than when the probe lies opposite to the peripheral portion of the breast. Similarly, the ultrasonic wave traveling from the position of the depth L to the probe has a lower intensity in the case of the tip portion than in the case of the peripheral portion. As a result, in a sectional image such as a C plane image which is parallel to a scanning plane (for example, an image at the depth L), a higher intensity (bright color) is expressed in the peripheral portion, whereas a lower intensity (dark color) is expressed in the tip portion. Such a decrease may lead to a reduced contrast in display images or a reduced accuracy of image analysis.

The present invention has been developed in view of the above-described problems. An object of the present invention is to provide a technique for an apparatus that acquires characteristics information on an object by allowing a probe to scan the object, while transmitting and receiving an ultrasonic wave to and from the object, the technique responding to changes in the amount of attenuation according to the position of the probe.

The present invention provides an object information acquiring apparatus comprising:

a receiver including a plurality of elements each transmitting an acoustic wave, receiving an echo wave resulting from reflection of the acoustic wave by an object, and outputting an electric signal;

a transmission controller that controls an intensity of the acoustic wave transmitted from each of the plurality of elements;

a scanner that moves the receiver in a predetermined scanning region; and

an information processor that acquires characteristics information on an inside of the object using the electric signal,

wherein the transmission controller controls the intensity of the acoustic wave in accordance with a shape of the object and a position of the receiver in the predetermined scanning region.

The present invention can provide a technique for an apparatus that acquires characteristics information on an object by allowing a probe to scan the object, while transmitting and receiving an ultrasonic wave to and from the object, the technique responding to changes in the amount of attenuation according to the position of the probe.

Further features of the present invention will become apparent from the following description of exemplary embodiments with reference to the attached drawings.

### BRIEF DESCRIPTION OF THE DRAWINGS

FIG. 1 is diagram depicting a configuration of an object information acquiring apparatus;

FIG. 2 is a diagram depicting a configuration of a signal processor;

FIG. 3 is a diagram depicting a distance between a probe and an object and an object thickness from the object to any C plane;

FIGS. 4A, 4B, and 4C are diagrams depicting the shapes of an applied voltage and a transmitted ultrasonic wave in a transmission controller;

FIGS. 5A, 5B, 5C, and 5D are diagrams depicting timings for selection of a conversion element and voltage application and the shape of the transmitted ultrasonic wave;

FIGS. 6A and 6B are diagrams depicting the number of transmitted pulses and the shape of a transmitted ultrasonic signal;

FIG. 7 is a diagram illustrating an example in which a transmission focus position is changed according to a probe position;

FIG. 8 is a diagram depicting a configuration of a variation of Embodiment 2;

FIG. 9 is a diagram depicting a configuration of another variation of Embodiment 2;

FIGS. 10A and 10B are a diagram depicting a configuration of Embodiment 4;

FIGS. 11A, 11B, and 11C are diagrams illustrating the use of a convex probe and bowl-shaped probes; and

FIG. 12 is a diagram depicting a configuration of a transmission controller.

### DESCRIPTION OF THE EMBODIMENTS

Preferred embodiments of the present invention will be described below with reference to the drawings. However, dimensions, materials, shapes, relative arrangements, and the like of components described below should be changed as needed according to a configuration of an apparatus to which the present invention is applied and various conditions. Hence, the dimensions, materials, shapes, relative arrangements, and the like of the components described below are not intended to limit the scope of the present invention to the following description.

The present invention relates to a technique for detecting an acoustic wave propagating from an object to generate and acquire characteristics information on the interior of the object. Hence, the present invention is considered to be an object information acquiring apparatus or a method for controlling the object information acquiring apparatus, or an object information acquiring method or a signal processing method. The present invention is also considered as a program that allows an information processing apparatus including hardware resources such as a CPU to execute these methods, or as a storage medium that stores this program.

An object information acquiring apparatus in the present invention includes an apparatus that utilizes an ultrasonic echo technique and that transmits an ultrasonic wave to an object and that receives a reflected wave (echo wave) reflected inside the object to acquire object information in form of image data. For the apparatus utilizing the ultrasonic echo technique, the object information acquired is information reflecting a difference in acoustic impedance among tissues inside the object.

The acoustic wave referred to in the present invention is typically an ultrasonic wave and includes a sound wave and an elastic wave. An electric signal into which an acoustic wave has been converted by a probe or the like is also referred to as an acoustic signal. However, description in the present specification in relation to the ultrasonic wave or the acoustic wave is not intended to limit the wavelength of the elastic wave thereof. An electric signal based on an ultrasonic echo is also referred to as an ultrasonic signal.

### Embodiment 1

### Apparatus Configuration

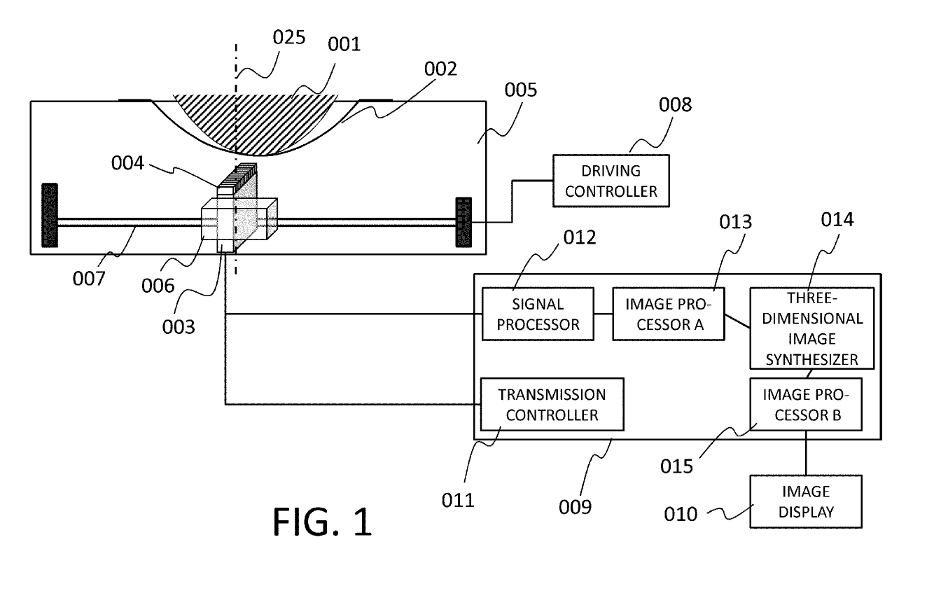

A configuration example of an ultrasonic echo apparatus according to the present invention will now be described with reference to FIG. 1. An object (for example, the breast) is denoted by reference numeral 001. A holding member that holds the object 001 is denoted by reference numeral 002. A probe that transmits an ultrasonic wave and detects an echo wave from the interior of the object is denoted by reference numeral 003. The probe 003 has a plurality of conversion elements 004. A matching material 005 is present between the probe 003 and the holding member 002 such that an acoustic wave propagates through the matching material 005. The probe 003 is fixed on a carriage 006. The carriage 006 is moved by a driving mechanism 007. A driving controller 008 serves to control the driving mechanism 007.

A system controller 009 creates a three-dimensional image from an image signal for the object 001 received by the probe 003 within a scan range. An image display 010 displays the three-dimensional image created by the system controller 009. The probe corresponds to a receiver in the present invention. The holding member corresponds to a holder in the present invention. The driving mechanism corresponds to a scanner in the present invention. The system controller corresponds to an information processor in the present invention.

The system controller 009 includes a plurality of units. A transmission controller 011 controls a driving timing for each of the conversion elements 004 corresponding to a focus position in order to adjust a transmission focus of an ultrasonic wave. A signal processor 012 reconstructs an ultrasonic echo signal from the object 001 into a two-dimensional image. An image processor A (013) executes image processing on the reconstructed image data. A three-dimensional image synthesizer 014 converts the reconstructed image into a three-dimensional image based on coordinates of the probe 003 driven by the driving mechanism 007 to perform scanning. An image processor B (015) executes image processing on the three-dimensional image data.

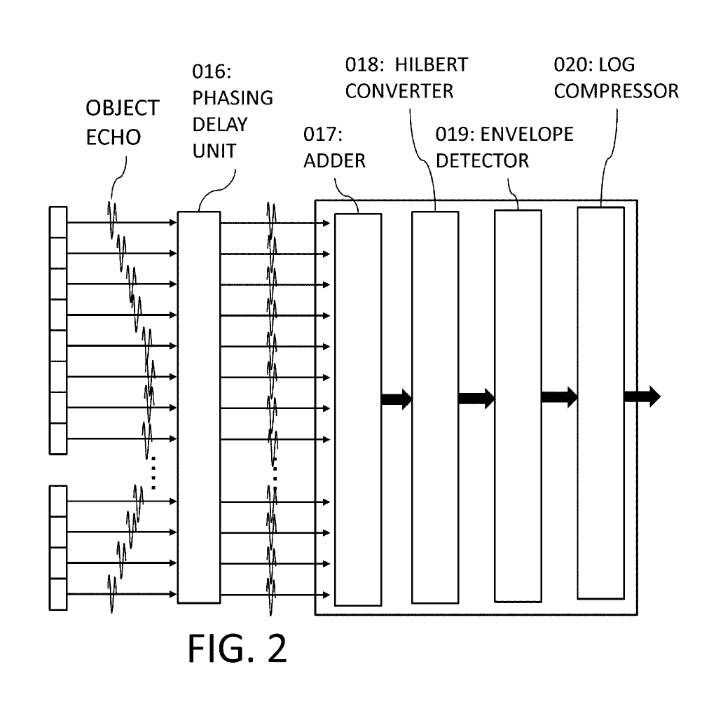

FIG. 2 depicts a configuration of the signal processor 012. A phasing delay section 016 adjusts phases of signals received by the conversion elements 004. An adder 017 adds together signals that have been subjected to a delay process. A Hilbert converter 018 executes Hilbert conversion on a signal resulting from the addition. An envelope detector 019 performs detection. An LOG compressor 020 performs LOG compression on the detected signal. The configuration of the signal processor is not limited to this and any configuration may be used long as the signal processor can perform amplification, digital conversion, correction, delay, or the like on electric signals output from the conversion elements.

(Functions of the System)

The system controller transmits an ultrasonic wave to the object 001 and converts an echo signal generated inside the object or on the surface of the object. The transmission controller 011 determines a delay time according to which a plurality of group of the conversion elements 004 forming a transmission aperture are driven in order to focus a transmission beam at a desired position (a position with respect to the probe in a ultrasonic transmission direction, that is, a depth). The transmission controller 011 sends driving signals to the conversion elements 004 based on the delay time. Then, the conversion elements 004 generate ultrasonic waves based on the driving signals and transmit the ultrasonic waves to the object 001.

The transmitted ultrasonic waves propagate through the matching material 005 and the holding member 002 to the object 001. Subsequently, echo waves reflected and scattered by the object 001 partly return to the conversion elements 004. A plurality of groups of conversion elements 004 forming a reception aperture receive and convert the echo waves into electric signals (reception signals). Amplification, correction, digital conversion, or the like is executed on the reception signals as needed.

The reception signals are reconstructed by the signal processor 012 into image data indicative of characteristics information. In FIG. 2, the phasing delay section 016 determines a delay time for reception signals based on an imaging position on an image scan line 025 in FIG. 1 and coordinate information on the positions of the conversion elements 004 forming the reception aperture. The phasing delay section 016 then executes the delay process on the reception signals. The image scan line means an area on a line in which an image is reconstructed by a reconstruction process.

The reception signals having been subjected to the delay process are added together by the adder 017. Subsequently, a resultant synthetic signal is subjected to Hilbert conversion and envelope detection by the Hilbert converter 018 and the envelope detector 019 to reconstruct an image. Besides the phasing addition method described herein, a reconstruction technique such as adaptive signal processing may be utilized. The reconstructed image data undergoes LOG compression by the LOG compressor 020 to complete image data on the image scan line 025. A series of processes is executed with the image scan line 025 moved to create a two-dimensional ultrasonic image data along a scan direction.

The image processor A (013) executes an edge emphasis process, a noise removal process, a contrast emphasis process, or the like on the created two-dimensional ultrasonic image data. Note that these types of image processing may be implemented later by the image processor B (015). The system controller executes the above-described processing on data obtained by the probe 003 transmitting and receiving ultrasonic waves while moving in a predetermined scanning region, to generate three-dimensional image data. After executing the three-dimensional-image acquisition process, the three-dimensional image synthesizer 014 arranges the three-dimensional image data in association with coordinate positions in the scanning region defined by the driving controller 008. The shape of the scanning region is not limited to a generally flat surface. The driving controller may move the probe in three-dimensional directions.

Instead of being executed after a B mode image is created, the three-dimensional-image acquisition process may be achieved by using the signal processor 012 to accumulate signals without processes carried out by components following the Hilbert converter 018 and using the three-dimensional image synthesizer 014 to execute a synthetic aperture process. The synthetic aperture process allows the resolution of images in a scanning direction of the probe 003 to be uniformized in the depth direction. Various other known techniques for obtaining three-dimensional image data may be utilized.

The image processor B (015) adjusts the created three-dimensional image data, for example, executes a sharpening process, a noise removal process, or the like. The image display 010 displays any sectional images. Image processing can be used to reduce brightness unevenness at the same depth that results from a variation in the amount of attenuation and that is a problem to be solved by the present invention. However, eliminating loss of image information is impossible. As the image display 010, a liquid crystal display, a plasma display, an organic EL display, or the like may be utilized. The image display 010 need not necessarily be a part of the apparatus. It is also preferable that the apparatus in the present invention create only image data and allow an external image display to display the image data.

(Driving of the Probe)

Each of the conversion elements 004 in the probe 003 converts an electric signal into an ultrasonic wave. Preferred conversion elements are piezoelectric elements such as PZTs, PVDF elements, cMUT elements, and the like which have relatively high conversion efficiency. The use of a probe with a plurality of conversion elements 004 one- or two-dimensionally arranged therein is expected to improve an SN ratio and to reduce measurement time. In the following description, transmission and reception of an ultrasonic wave are performed by a common conversion element. However, different conversion elements may be used for transmission and for reception, respectively.

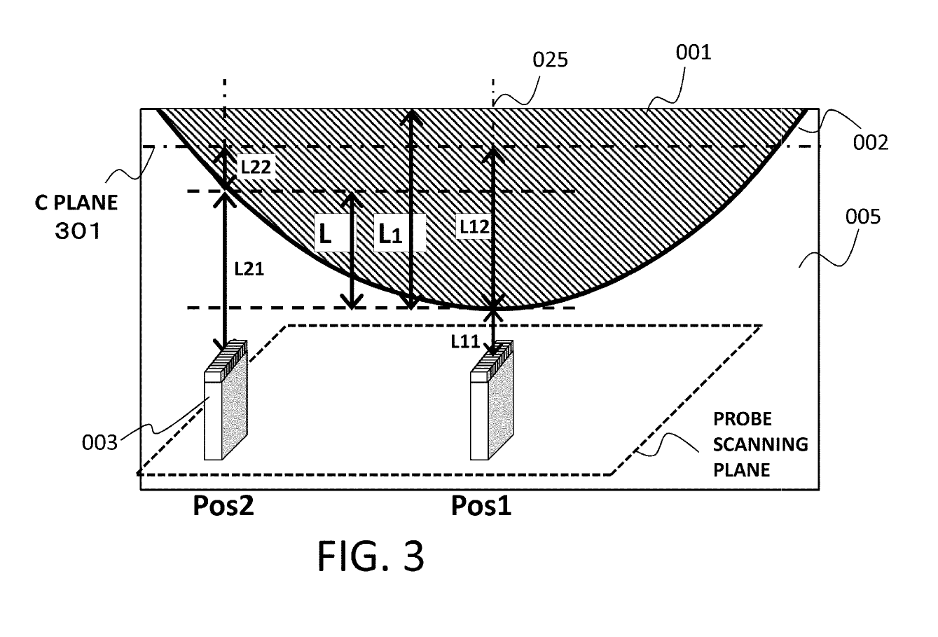

Driving of the probe 003 and an imaging method used during the driving will be described with reference to FIG. 3. A scanning region herein is a scanning plane shaped in the form of a generally flat surface. The probe 003 installed on the carriage 006 is moved by the driving mechanism 007 in the scanning plane opposite to the holding member 002. As the driving mechanism 007, for example, a combination of a pulse motor and a ball screw or a linear motor may be utilized. A rotation mechanism for the carriage 006 may be provided to tilt the probe 003 to any angle. As described below, the probe 003 may also be three-dimensionally moved. The three-dimensional movement and tilt of the probe allow ultrasonic waves to be obtained in various directions with respect to the object, providing accurate image data.

(Holding Member)

The use of the holding member 002 stabilizes the shape of the object to improve the calculation accuracy of calculations of the amount of attenuation and calculations for image reconstruction. Control time and the amount of calculation can be reduced by utilizing sound pressure control information pre-stored in a memory and corresponding to the shape of the holding member. However, the present invention is also applicable when the holding member 002 is not used.

The holding member 002 used is acoustic-wave transmissive. A material for the holding member 002 desirably involves a small difference in acoustic impedance between the object 001 and the matching material 005. In order to allow the object 001 to be suitably held, a rigid member or a stretchable member is preferably used. Examples of the rigid member include resin materials such as PET, polymethyl pentene, and acrylic. Examples of the stretchable member include rubber sheets of latex, silicone, and the like and materials such as urethane. Alternatively, a holding mechanism containing a combination of a plurality of materials may be used.

Preferably, the holding member 002 is interchangeably installed. When the breast is inserted into the apparatus through an opening in a housing, an installation portion may be provided which includes a bracket or a hook to allow the holding member to be easily fixed. This allows the holding member 002 to be easily changed according to the subject or the contents of measurement. Preferably, control information is pre-stored in the memory for each holding member to be changed.

The matching material 005 acoustically matches the object (or the holding member) with the probe. Therefore, the matching material 005 preferably allows acoustic waves to propagate through the matching material 005 and avoids preventing scanning by the probe 003. Examples of the matching material 005 include liquids such as water, DIDS, PEG, silicone oil, and castor oil.

(Acoustic Attenuation Difference)

In many cases, the object 001 is shaped to have a curvature or unevenness. For example, for the breasts, a central portion protrudes with respect to a peripheral portion. In contrast, the object may be shaped such that the central portion is depressed with respect to the peripheral portion as in the case of the buttocks and the arch of the foot. In an example in FIG. 3, the scanning plane of the probe 003 is not parallel to the surface of the object 001. In FIG. 3, a C plane 301 substantially parallel to the probe scanning plane is to be displayed. When the probe is located at Pos1, in a normal direction of the scanning plane, a distance L11 is present from the probe to the object surface, and a distance L12 is present from the object surface to the C plane. When the probe is at Pos2, a distance L21 is present from the probe to the object surface, and a distance L22 is present from the object surface to the C plane. Thus, an in vivo passage distance and a matching material passage distance on the path of an ultrasonic wave and the ratio between these distances vary according to the position of the probe. In general, an ultrasonic wave attenuation rate is higher in the living organism than in the matching material. Consequently, both the transmitted ultrasonic wave and the echo wave are more likely to attenuate at Pos1. As a result, the value of brightness in the C plane varies.

With a large difference in brightness within the C plane, the degree of reproduction of images of the interior of the object may decrease in image display, particularly in real-time display. For example, the image data contained in certain C plane image data is assumed to have a brightness varying between “0 and 100”. Given that a manipulator adjusts the range of the brightness of images displayed on the display to between “20 and 80”, information is lost which concerns pixels with brightness values falling outside the range. Therefore, particularly in ultrasonic apparatuses that display images in real time, the accuracy of image analysis may decrease.

Such problems caused by a variation in output value will be described below in further detail. For example, in image data at a C plane position corresponding to Pos1, the output value is multiplied by a larger gain in order to correct attenuation during in vivo propagation over a long distance. However, application of this condition to Pos2 may lead to an excessive gain to the output value. Specifically, amplification is performed by the value of the product of three numerical values including a distance difference L, the acoustic attenuation characteristics of the object 001, and an ultrasonic frequency during transmission and reception. As a result, the gain reaches several tens of dB depending on conditions and may exceed an upper limit of a dynamic range. In contrast, when the image data at the C plane position corresponding to Pos1 is imaged under conditions set to allow image data at a C plane position corresponding to Pos2 to be displayed, the amplification may be insufficient and signal intensity may be lower than the noise level of the apparatus.

(Preferred Acoustic Attenuation Characteristics of the Holding Member)

To avoid this phenomenon, the adverse effect of an acoustic attenuation difference equivalent to a distance difference L needs to be reduced. In the present invention, control by the transmission controller 011 is changed to suppress the adverse effect of the acoustic attenuation difference. Specifically, the difference in acoustic attenuation between the object 001 and the matching material 005 that is equivalent to the length L [cm] is reflected in a transmitted sound pressure intensity (acoustic radiant intensity).

For example, water that does not substantially attenuate acoustic waves is used as the matching material 005. The attenuation characteristic of the object 001 is assumed to be 0.3 [dB/MHz/cm], and a central frequency of a signal processed by the signal processor 012 is set to 7 MHz. Then, a difference of approximately 4.2L [dB] occurs between the amount of attenuation at Pos1 and the amount of attenuation at Pos2. Thus, the transmission controller 011 makes adjustment so as to set the difference in transmitted sound pressure between Pos1 and Pos2 to 4.2L [dB/MHz] to enable a reduction in a difference in output value at the C plane. A transmitted sound pressure associated with the attenuation amount is set for positions between Pos1 and Pos2 and other positions on the scanning plane.

Such adjustment is performed as needed in accordance with the difference in acoustic attenuation characteristic between the object 001 and the matching material 005 and the distance that acoustic waves propagate through the object. In general, the object 001 has a higher level of acoustic attenuation characteristics than the matching material 005. Thus, the transmitted sound pressure intensity may be set to a large value at a position where the probe 003 is proximate to the object 001 and may be reduced proportionally with an increase in the distance between the probe 003 and the object 001.

Due to the characteristics of human bodies, many objects 001 are round in shape and are likely to protrude at a central portion. Thus, the intensity is effectively increased when the probe 003 is at a position corresponding to the central portion of the object and reduced when the probe 003 is at a position corresponding to the peripheral portion of the object. More specifically, when the scanning plane is shaped in the form of a generally flat surface, the transmitted sound pressure intensity is reduced proportionally with an increase in the distance from the scanning plane to the holder in the normal direction of the scanning plane. In contrast, the transmitted sound pressure intensity is increased proportionally with a decrease in the distance from the scanning plane to the holder in the normal direction of the scanning plane.

Calculation of the acoustic attenuation characteristics needs the following five pieces of information.

(Information 1-1) acoustic attenuation characteristics of the matching material 005: α2 [dB/MHz/cm]
(Information 1-2) acoustic attenuation characteristics of the object 001: α1 [dB/MHz/cm]
(Information 1-3) shape of the object 001
(Information 1-4) scanning trajectory of the probe 003
(Information 1-5) frequency of signals processed by the signal processor 012: f1 [MHz]

The information 1-1, the information 1-4, and the information 1-5 are known from settings for the system and materials used. On the other hand, the information 1-2 and the information 1-3 involve significant variations among tissues and large differences among individuals and are thus preferably set with reference to experimental values and literature values or acquired through pre-scanning.

The information 1-2 is preferably specified such that, when the object 001 is the breast, α1=0.3 to 0.8 [dB/MHz/cm]. The breast is characterized in that young people tend to have many mammary gland layers and that the rate of fat tends to increase with age. The mammary gland layer has a higher level of acoustic attenuation characteristics than the fat layer, and thus, the level of the acoustic attenuation characteristics of the breast may be increased proportionally with a decrease in age.

(Determination of the Object Shape)

When the shape of the object 001 can be predetermined using any technique, distance information on the probe 003 and the object 001 can be pre-calculated for each position on the scanning plane. In that case, the distance information for each coordinate of the probe 003 and control parameters based on the distance information are saved to the memory. The transmission controller 011 references the memory based on the coordinate of the probe 003 to enable easy acquisition of the distance information or transmission control information used to change the transmitted sound pressure intensity.

When the object 001 is a soft tissue such as the breast, a rigid member is preferably used as the holding member in order to accurately obtain the information 1-3 (shape). The shape of the holding member 002 preferably fits the object 001. For example, for the breast, the holding member 002 is shaped in the form a cup. The use of a rigid member allows the holding shape of the object 001 to be defined enabling the distance between the probe 003 and the object 001 to be set, whereby the information 1-3 can be easily obtained.

Meanwhile, even when a stretchable material is selected as the holding member, the holding shape can be estimated to some degree based on the hardness and film thickness of the holding member, information on the object, and so on. The information on the object includes, in the case of the object being the breast, size information such as a cup size, a topbust size, and an underbust size and subject information such as race, age, and body conditions. When the object 001 is the breast, the breast is difficult to squeeze when the subject is young and has many mammary gland layers or when the subject has a period. These pieces of information are used to customize the holding member 002 to allow the holding shape to be more accurately estimated. Even when a stretchable holding member is used, the amount of protrusion (L1) of the object 001 can be kept small by increasing the hardness or film thickness of the holding member 002 or pre-tensioning the holding member 002.

One method involves executing image taking using a camera or pre-scanning before production imaging (reception of an ultrasonic wave and generation of an echo image) to acquire the object shape and calculating the distance between the probe 003 and the object 001. A method of acquiring the object shape through scans immediately before the production imaging is effective depending on the holding aspect of the object. These techniques will be described below in detail.

(Transmission Controller)

Based on the object shape determined above, a difference in in vivo propagation path length (L in FIG. 3) between Pos1 and Pos2 is determined. Based on the difference, the difference in transmitted sound pressure intensity between Pos1 and Pos2 can be determined. Control elements for the transmitted sound pressure intensity in the transmission controller 011 are the following pieces of information.

(Information 2-1) transmitted sound pressure amplitude value
(Information 2-2) number of transmission aperture elements
(Information 2-3) number of transmitted pulses
(Information 2-4) transmission frequency

Among these pieces of information, the transmitted sound pressure amplitude value (information 2-1) is most suitable. Changing only the amplitude value changes an S/N ratio alone. This makes image quality at Pos1 similar to image quality at Pos2 in the image, allowing C plane images to be easily matched with one another. On the other hand, the other items cause a change in the shape of a transmitted beam, changing the atmosphere of the image including resolution.

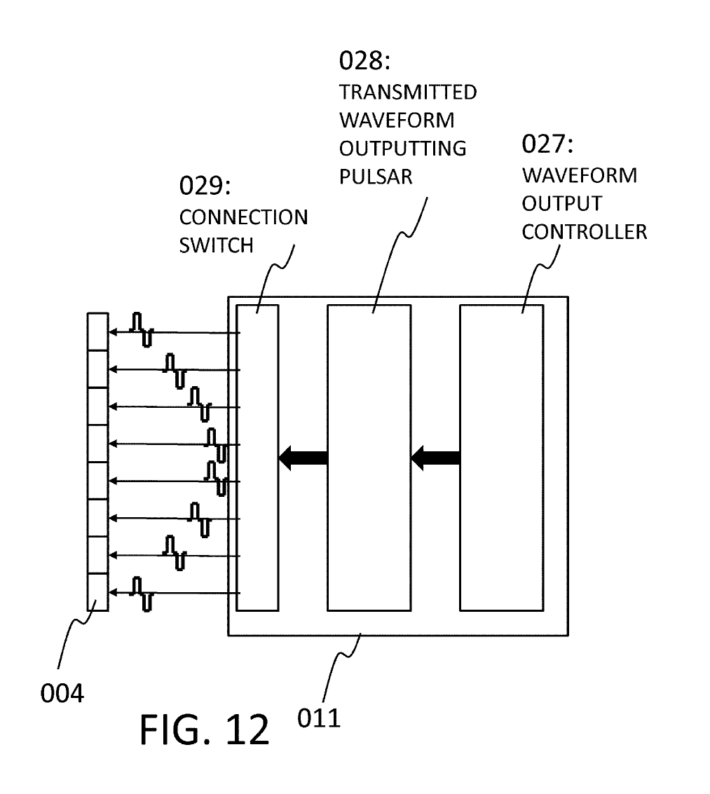

The techniques will be described. As depicted in FIG. 12, the transmission controller 011 includes a waveform output controller 027, a transmitted waveform outputting pulsar 028, and a connection switch 029. The waveform output controller 027 controls a pattern of a transmitted waveform. The transmitted waveform outputting pulsar 028 applies voltages to the conversion elements 004 in accordance with a command from the waveform output controller 027. The connection switch 029 allots analog signals from the transmitted waveform outputting pulsar 028 to the respective conversion elements 004.

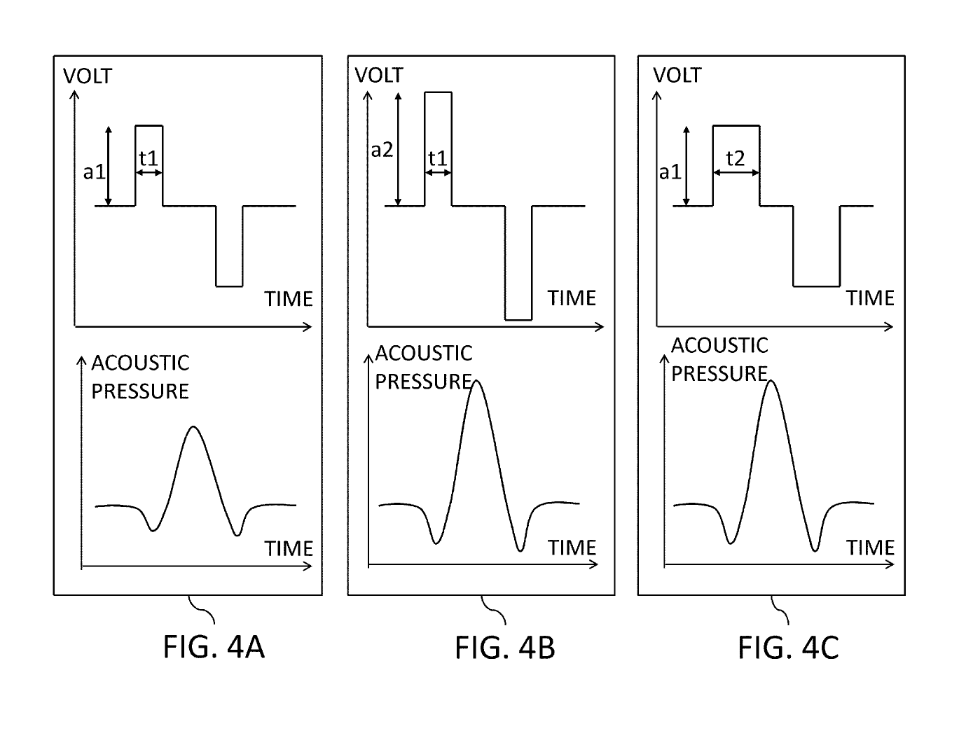

In FIGS. 4A to 4C, an upper graph represents the pulse of an electric signal applied to each of the conversion elements 004 by the transmission controller 011. The axis of abscissas indicates time, and the axis of ordinate indicates an applied voltage value. In FIGS. 4A to 4C, a lower graph indicates an ultrasonic signal output by the conversion element 004 in accordance with each pulse. The axis of abscissas indicates time, and the axis of ordinate indicates a sound pressure intensity. Techniques for changing the amplitude value of the ultrasonic wave include a technique for changing the applied voltage value and a technique for changing an applied pulse width.

For example, a comparison between FIG. 4A, serving as a reference, and FIG. 4B indicates that the transmitted sound pressure amplitude value increases as the applied voltage value increases from a1 to a2. Furthermore, a comparison between FIG. 4A and FIG. 4C indicates that the transmitted sound pressure amplitude value increases as the applied pulse width increases from t1 to t2. The applied voltage value or the applied pulse width is adjusted by the transmitted waveform outputting pulsar 028 having received a command from the waveform output controller 027. This control technique is characterized by involving substantially no change in the shape of the ultrasonic wave but only a change in amplitude value. Any waveform generator may be used instead of the transmitted waveform outputting pulsar 028.

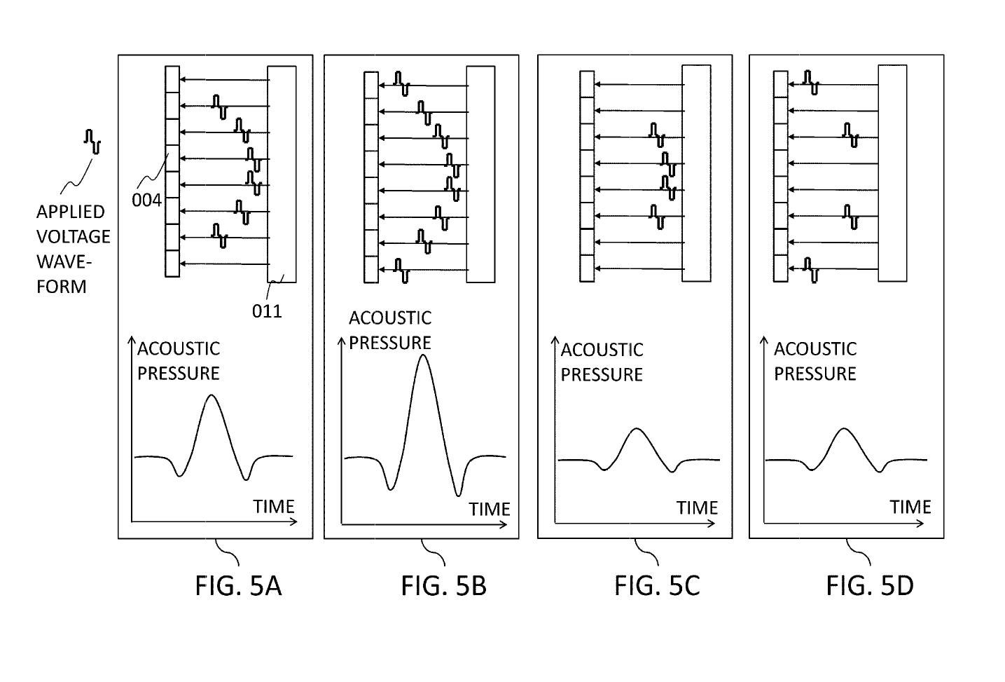

Now, control based on the number of transmission aperture elements (information 2-2) will be described using FIGS. 5A, 5B, 5C, and 5D. The upper sides of FIGS. 5A, 5B, 5C, and 5D illustrate examples of transmission control that differ in the positions and number of the conversion elements 004 included in an aperture element group and in voltage application timings. In FIGS. 5A, 5B, 5C, and 5D, the lower side illustrates the sound pressure waveform of an ultrasonic wave corresponding to each type of transmission control. FIGS. 5A, 5B, 5C, and 5D illustrate a probe with eight conversion elements 004 linearly arranged therein. The intensity in FIG. 5B, depicting more aperture elements than FIG. 5A, serving as a reference, is higher than the intensity in FIG. 5A. In FIG. 5C, depicting less aperture elements, the intensity is lower. To perform such driving, the connection switch 029 is controlled to change a combination of the conversion elements 004 to which voltages are to be applied. When the matching material 005 contains water, preferably the control in FIG. 5B is selected for Pos1 and the control in FIG. 5C is selected for Pos2.

However, FIG. 5C involves a smaller transmission aperture width than FIG. 5B, and thus, the resolution at Pos2 is lower than the resolution at Pos1. Thus, the conversion elements 004 may be selected as depicted in FIG. 5D rather than in FIG. 5C. This increases the aperture width to make the resolution uniform.

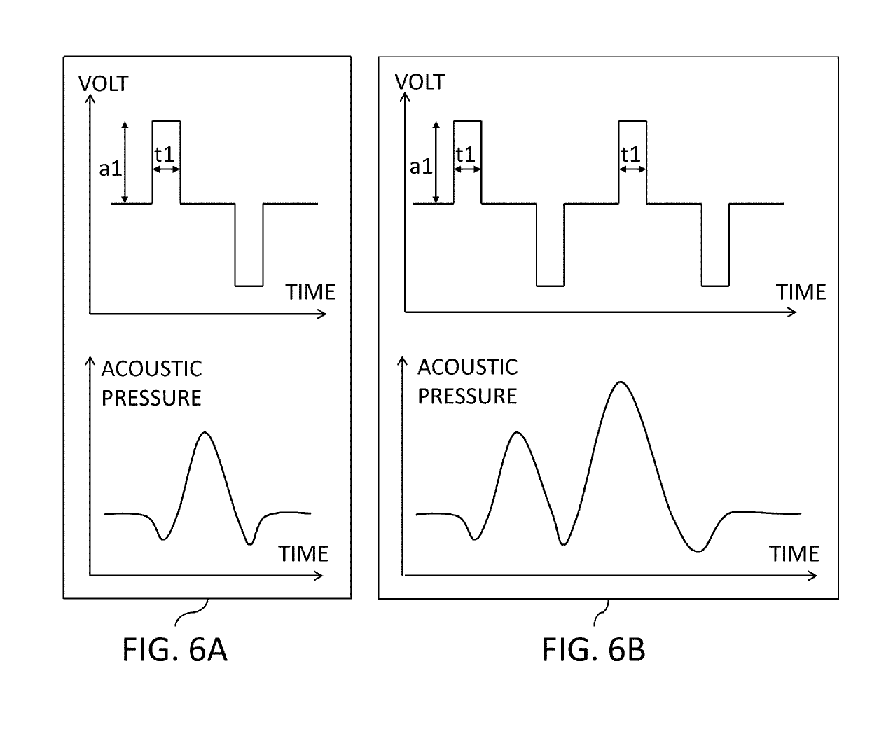

Now, a technique using the number of transmitted pulses (information 2-3) will be described using FIGS. 6A and 6B. FIG. 6A illustrates that voltages are applied using one positive pulse and one negative pulse as in the case of FIG. 4A. On the other hand, FIG. 6B illustrates that two positive pulses and two negative pulses are applied by repeating twice the pulse application in FIG. 6A, serving as a reference. Transmission energy is increased by an increased number of applied pulses as in FIG. 6B. When a plurality of pulses is applied as depicted in FIG. 6B, the second and subsequent pulses of a transmitted ultrasonic wave may have a larger amplitude value than the first pulse of the transmitted ultrasonic wave, though this may depend on the characteristics of the conversion elements 004, the pulse width of the applied voltage, and the like. When the matching material 005 contains water, preferably the control in FIG. 6B is selected for Pos1 and the control in FIG. 6A is selected for Pos2.

However, an increase in the number of transmitted pulses reduces the resolution in the depth direction (time direction) of an ultrasonic image. Thus, the number of waves is preferably increased or reduced to the extent that degradation of the resolution is invisible.

Now, a technique of changing the transmission frequency (information 2-4) to suppress the output value difference will be described. In general, ultrasonic waves are characterized in that a transmitted wave with a lower frequency is more unlikely to be attenuated. Thus, an ultrasonic wave with a relatively low frequency is transmitted at Pos1, and an ultrasonic wave with a relatively high frequency is transmitted at Pos2. However, each frequency needs to be determined taking the frequency characteristics of the conversion elements 004 into account. That is, when the frequency of the ultrasonic wave is changed, the sensitivity of the conversion elements 004 at each frequency needs to be taken into account in addition to the voltage and the pulse width of the applied pulse. When the transmission frequency (information 2-4) is changed, the change is preferably used in conjunction with the adjustment of the (information 2-1), the (information 2-2), and the (information 2-3).

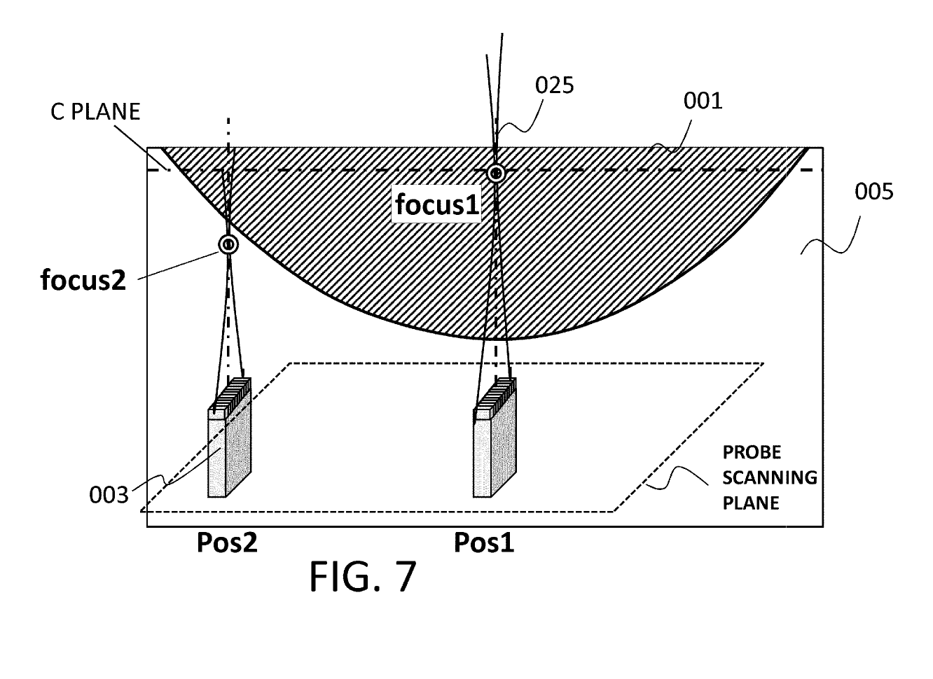

Another control method for suppressing the output value difference at any section is to change a transmission focus position according to a place as depicted in FIG. 7. Normally, the sound pressure is maximized at the transmission focus position and decreases with increasing distance from the focus position. Thus, as depicted in FIG. 7, a deep focus position (focus1) is set for Pos1, where the object is thick, and a shallow focus position (focus2) is set for Pos2.

However, in the methods other than the method using the transmitted sound pressure amplitude value (information 2-1), the resolution varies within any section, and thus, conditions are preferably set with a variation in resolution taken into account. Furthermore, achievable transmission conditions are limited by the performance of the transmitted waveform outputting pulsar 028. Thus, when the control based only on the transmitted sound pressure amplitude value is difficult, a combination with another control technique is effective. However, the above-described control techniques may be optionally combined together in accordance with the configuration and performance of the apparatus, the conditions of the object, and the like.

(Configuration of the Image Processor)

The output value difference at any section can be reduced by changing the control by the transmission controller 011 in association with the coordinate position of the probe 003 to change the intensity of the transmitted ultrasonic wave, as described above. The image processor B (reference numeral 015) adjusts the output value to further reduce the output value difference, allowing suppression of uneven brightness.

When the transmitted sound pressure intensity is changed using the above-described method, the intensity of reflected echo wave is also changed. Basically, at a probe position with a high degree of attenuation, the transmitted sound pressure also increases. Consequently, the intensity of the echo wave is expected to be increased. However, attenuation of the transmitted wave or the echo wave is preferably corrected using the gain of a reception signal or the like depending on the shape of the object or acoustic propagation characteristics.

(Variation of the Probe)

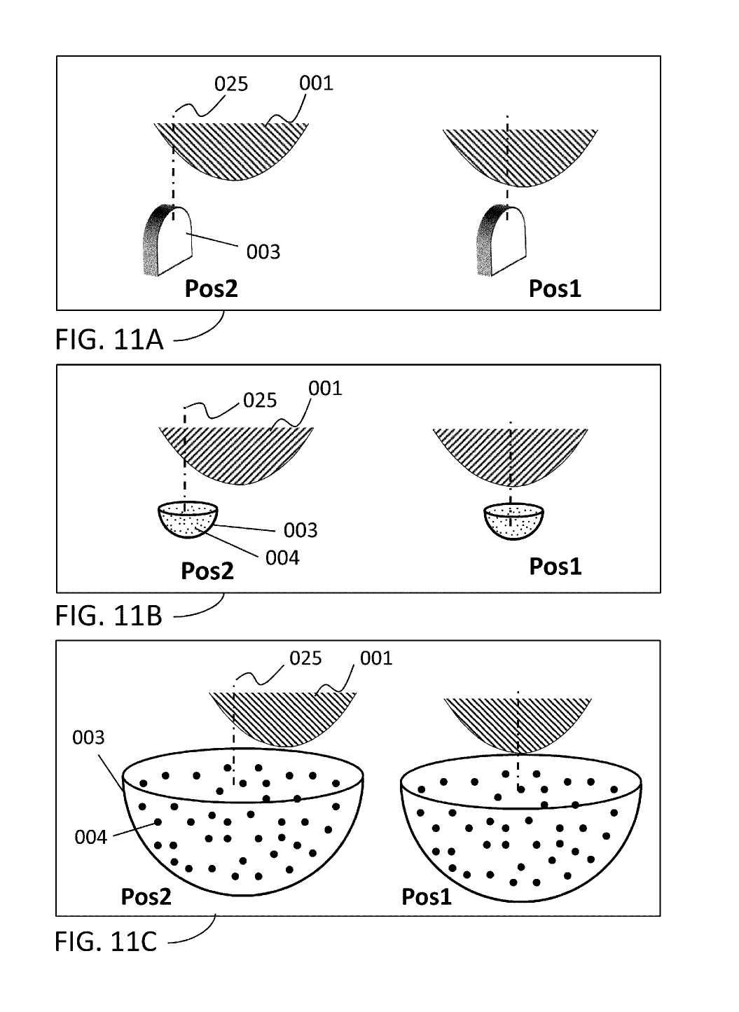

The present invention is applicable to an apparatus including any of various such probes as depicted in FIGS. 11A, 11B, and 11C instead of a 1D probe or a 2D probe. For example, FIG. 11A depicts a convex probe with the conversion elements 004 arranged therein so as to have a curvature. FIGS. 11B and 11C depict a large and a small bowl-shaped probes with conversion elements arranged on a hemispherical surface. The present invention is effective even on these probes because an output value difference results from the distance from the position of the group of conversion elements 004 forming the transmission aperture or the reception aperture to the object surface on the image scan line 025.

A probe with conversion elements arranged on a bowl-shaped support member can receive, in various directions, acoustic waves propagating from the object, improving the accuracy of reconstructed images. In the bowl-shaped probe, the conversion elements fail to have the same high-sensitivity direction. Consequently, when the holding member is partitioned into certain regions, the positions of the regions are precluded from being specified in association with the high-sensitivity directions of the conversion elements. On the other hand, the bowl-shaped probe is provided with a high-sensitivity area (high-resolution area) where the high-sensitivity directions of a plurality of elements concentrate. Thus, when positions in the holding member are identified, the positions can be specified in association with the high-sensitivity area.

In the above-described method, even when the degree of attenuation of the transmitted or received ultrasonic wave varies according to the position of the probe moving on the scanning region because the object is shaped to have a depressed portion or a protruding portion, the transmitted sound pressure is controlled according to the position. This enables a reduction in a variation in the intensity of the acoustic signal and in the output value of image data.

### Embodiment 2

A system configuration of an ultrasonic echo apparatus according to Embodiment 2 described below is basically similar to the system configuration in FIG. 1. In the following description, the same components are denoted by the same reference numerals. The system controller 009 in the present embodiment includes a memory 022 that is a storage medium connected to the transmission controller 011 and enabled to transmit and receive information. The suitable object 001 in the present embodiment is one breast.

In the present embodiment, a 1D linear probe with 256 channels is used as the probe 003. The conversion elements 004 forming the probe 003 are PZTs having a central frequency of 7 MHz and an element size of 4 mm and arranged at a lateral element pitch of 0.2 mm. As the holding member 002, a cup-shaped member is adopted which is formed of PETG and which has a thickness of 0.5 mm. The holding member 002 sets the protruding distance of the breast to 30 mm from the chest wall. The driving mechanism 007 for the probe 003 is installed so as to set the minimum distance between the holding member 002 and the probe 003 to 10 mm.

The matching material 005 and was used while being circulated by a pump. In the present embodiment, water temperature was kept at approximately 35° C. using a heater. Keeping the water temperature in this manner is effective for preventing the subject from feeling uncomfortable and for defining a sound velocity in the matching material 005 to improve the accuracy of image reconstruction.

Methods for the following are similar to the corresponding methods in Embodiment 1: control of transmitted ultrasonic waves including electronic scanning, reception of echo waves, processing of reception signals, mechanical scanning by the probe 003, an image reconstruction process using reception signals, and the like. First, the transmission controller 011 transmits an electric signal with a timing therefor controlled to focus an ultrasonic wave on a desired position, to each of the conversion elements 004. Each conversion element 004 transmits an ultrasonic signal to the object 001. The central frequency of the ultrasonic signal is adjusted to 7 MHz.

In the present embodiment, the holding member 002 lying opposite to the scanning region protrudes at the center of the holding member 002 so as to conform to the shape of the breast. Thus, the scanning region for the probe 003 was divided into a central, first region and a peripheral, second region according to the distance from the scanning plane to the holding member 002 in a normal direction. For the first area, the transmission controller 011 sets the transmission focus position at distances of 20 mm and 40 mm from the probe 003 to reconstruct images by two-stage focus processing. For the second area, the transmission controller 011 sets the transmission focus position at a distance of 40 mm from the probe 003 to reconstruct images by one-stage focus processing. Thus, the transmission focus setting is changed according to the positions of the object 001 and the probe 003 to avoid setting the transmission focus for regions in which the object 001 is not present. This enables a reduction in imaging time.

For both the first and second regions, the number of conversion elements 004 is set to 64 under the transmission condition indicating that the focus is placed at a distance of 40 mm. The transmitted sound pressure amplitude value for the second area was set to approximately 10% of the corresponding value for the first region under the condition that the focus was placed at a distance of 40 mm. This amount of change is calculated for Pos2 in accordance with Expression (1) using Pos1 in FIG. 3 as a reference.

2×L×(α1−α2)×f1 [dB] (1)

The value corresponding to the above-described 10% is determined to be 16.8 [dB] by substituting α1=0.4 [dB/MHz/cm], α2=0 [dB/MHz/cm], and f1=7 [MHz] into Expression (1).

This adjustment is performed by controlling the values of the voltages applied to the group of the conversion elements 004 and the applied pulse width. Control values are set for each coordinate position of the probe 003 and recorded in the memory 022. When the shapes of the breast and the holding member are known, the control values may be pre-acquired and recorded in the memory 022.

In the present embodiment, only the ultrasonic transmitted sound pressure is changed while the number of apertures, the focus position, and the transmission frequency are kept the same. As a result, the transmitted beam shape on the C plane to be observed is subjected to only few changes, reducing a variation in resolution within the reconstructed C plane image to allow uniform images to be realized. Thus, in the present embodiment, the output value difference within the C plane image is corrected to make images easy to see, reducing degradation of image quality of any section.

(Variation 1)

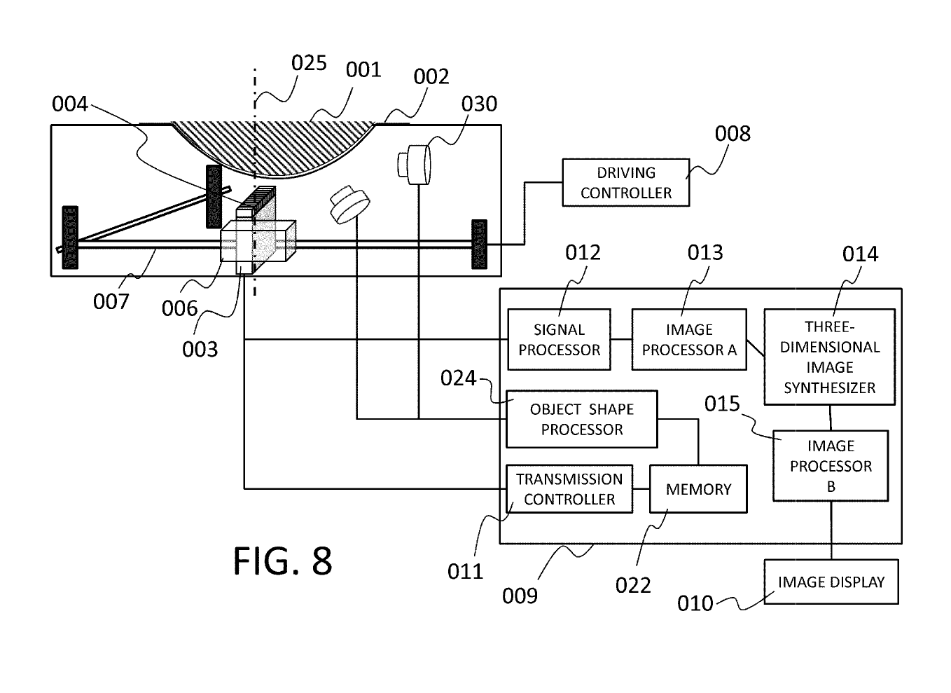

A shape acquisition method used when the shape of the object or the holding member is not known will be described using a block diagram in FIG. 8. In FIG. 8, two cameras 030 are arranged at side surfaces of a water tank. After the breast is fixed to the holding member 002, the cameras 030 take images the breast in a plurality of directions. Upon receiving the camera images, an object shape processor 024 calculates a three-dimensional shape of the breast, sets transmission conditions for each coordinate of the probe 003, and records the transmission conditions in the memory 022. This technique allows the three-dimensional shape of the object 001 to be calculated in a short time. The accuracy and speed of the shape acquisition are improved by increasing the number of cameras so as to allow images of the object 001 to be taken in various directions. Furthermore, images of the breast may be taken with one of the cameras moved. The object shape processor 024 executes various known image processing methods in accordance with programs or the like using an information processing resources such as a CPU.

(Variation 2)

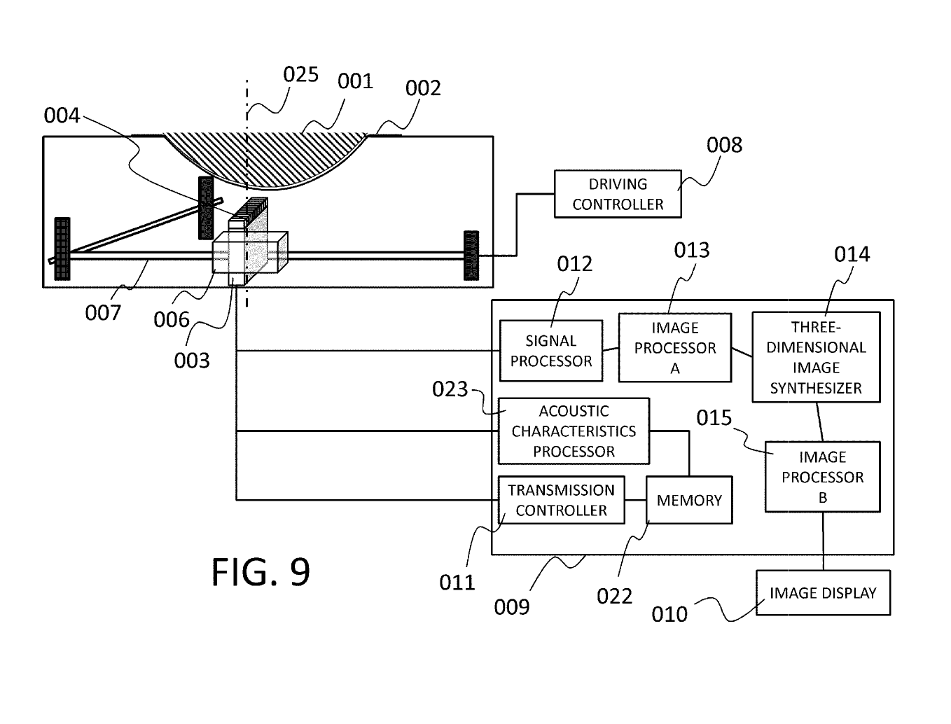

Another object shape acquisition method will be described using FIG. 9. An apparatus in this variation includes an acoustic characteristics processor 023 configured to the object shape using the results of pre-scanning executed before the production imaging. The acoustic characteristics processor 023 is connected to the probe 003 and the memory 022. For the pre-scanning, the one-stage focus processing with the transmission focus position set at a distance of 40 mm is adopted. The transmission conditions are not varied according to the position of the probe 003. The reason for the adoption of the given transmission conditions and the one-stage focus is that the purpose of the pre-scanning is simply to acquire the object shape. This variation is suitable when the holding member 002 is not used and when the holding member is flexible.

The acoustic characteristics processor 023 calculates a breast surface position based on acoustic signals obtained by the pre-scanning to acquire the three-dimensional shape of the breast. The breast surface position can be calculated using points of time when echo waves are generated as a result of the difference in acoustic impedance between the matching material and the living organism. That is, when ultrasonic waves are transmitted and received, a position where the first strong echo signal is detected corresponds to the surface of the object 001. To shorten the time for the pre-scanning, the number of set image scan lines 025 may be reduced.

Even when the object 001 is not in close contact with the holding member 002, strong echo signals from the interface of the holding member 002 can be suppressed by selecting, as the holding member 002, a member with an acoustic impedance close to the acoustic impedance of the matching material 005 as the holding member 002. As a result, echo signals from the surface of the object 001 are more easily extracted. This shape measuring method is suitable when the holding member and the matching material 005 are latex and water, respectively, and when the holding member and the matching material 005 are silicone rubber and silicone oil, respectively.

During the pre-scanning, the acoustic attenuation characteristics of the breast are effectively calculated based on a variation in the S/N ratio of the reception signal according to the depth so as to be referenced during setting of the transmission conditions. Based on these pieces of information, the transmission conditions are set for each coordinate of the probe 003 on the scanning region and recorded in the memory 022. This technique allows the breast position to be actually measured for each coordinate of the probe 003 and also allows the acoustic attenuation characteristics of the breast to be predetermined. Consequently, suitable transmission conditions can be set.

### Embodiment 3

In the description of Embodiment 3, the object 001 is difficult to hold and the shape of the object constantly changes. An apparatus configuration in the present embodiment is substantially similar to the configuration of the apparatus in the above-described variation 2, but does not require the memory 022 to have a function to save pre-scanning results.

When a thin film (for example, a latex sheet) is used as the holding member 002 to hold a soft object such as the breast, the object shape is constantly changed by body motion. Thus, even when the object shape is measured and recorded based on pre-scanning results or camera images, the resultant object shape is different from an object shape resulting from the production imaging. Thus, in the present embodiment, the acoustic characteristics processor 023 calculates the distance between the object 001 and the probe 003 in real time.

In the present embodiment, a 1D linear probe is used as the probe 003, and electronic scanning (linear scanning) is executed at each position to which the probe has been moved by mechanical scanning, to acquire a two-dimensional image. Then, immediately before execution of the linear scanning, ultrasonic waves are transmitted and received between the probe 003 and the object 001 to measure the distance. Transmission conditions used immediately before the linear scanning are the same as the transmission conditions for the above-described pre-scanning and are not changed according to the coordinate of the probe 003. The acoustic characteristics processor 023 processes electric signals generated by the probe having received echo waves from the breast surface to calculate the distance to the object. The acoustic attenuation characteristics of the breast are effectively calculated based on a variation in the S/N ratio of the reception signal according to the depth so as to be referenced for the transmission conditions. Based on these pieces of information, the transmission controller 011 calculates the transmission conditions for the probe 003 to drive the group of conversion elements 004.

In the technique of acquiring the distance between the probe and the object immediately before the linear scanning as in the present embodiment, the distance obtained immediately before the production imaging can be calculated. The present embodiment thus enables setting of more accurate transmission conditions than Embodiment 1. The present embodiment is particularly effective when the object is likely to be deformed and when the shape pre-acquired by the pre-scanning or the like is different from the shape acquired during the production imaging. Compared to the method with the pre-scanning, this method is advantageous in that the total time needed for the measurement is short and in that the distance information involves few errors because of the lack of time lag between the pre-scanning and the production imaging.

### Embodiment 4

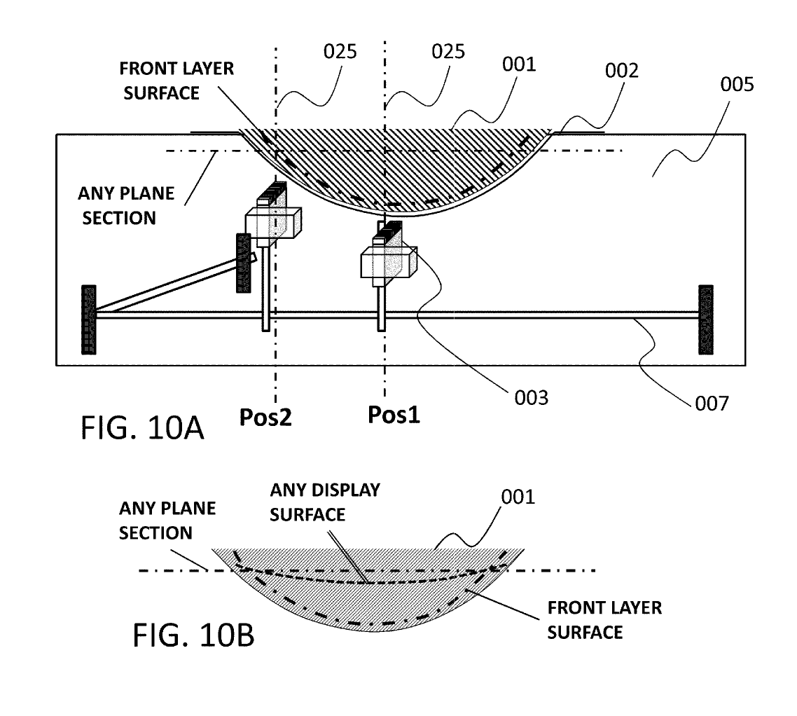

The configuration and basic operation of an apparatus in the present embodiment are the same as the configuration and basic operation in Embodiment 1. A difference between the present embodiment and Embodiment 1 is that the driving mechanism 007 that enables tri-axial movement is used to three-dimensionally move the probe 003 as depicted in FIG. 10A. In the present embodiment, the probe is three-dimensionally driven along the shape of the holding member 002 to enable the distance of the matching material 005 between the object 001 and the probe 003 to be minimized.

As the holding member 002, a cup-shaped member is adopted which is formed of PETG and which is 0.5 mm in thickness. The driving mechanism 007 drives the probe 003 along the shape of the holding member 002 to allow images of the object to be acquired. As a result, the present embodiment involves fewer changes in the distance between the object 001 and the probe 003 than Embodiment 1. Furthermore, the distance of the matching material 005 between the probe 003 and the object 001 is reduced all over an imaging region. Thus, a front layer surface of the object 001 exhibits high image quality and is even. However, the distance between the probe 003 and the object 001 may fail to be made completely constant during the mechanical scanning depending on the unevenness of the shape of the object 001 or the holding member 002 or the performance of the driving mechanism 007. Thus, in the present embodiment, the control values for the transmission controller 011 and output values from an image processor B (015) are preferably adjusted to enhance or uniformize image quality as in the case of Embodiments 1 and 2.

When the apparatus in the present embodiment is used to display an image of any plane section, the transmission controller 011 changes the transmitted sound pressure control value in accordance with the distance from the surface of the object 001 to the plane section on the image scan line 025. In FIGS. 11A, 11B, and 11C, the object 001 distance from the probe 003 to the any flat section is shorter at Pos 2 than at Pos1. Thus, the transmitted sound pressure is reduced at Pos2. To make the resolution uniform within the plane section, the number of conversion elements 004 to be driven is preferably changed between Pos1 and Pos2 to change the width of the transmission aperture. For example, in FIG. 10A, the aperture width at Pos1 is set larger than the aperture width at Pos2.

The present embodiment is applicable not only to a case where an image in any plane section is displayed but also to a case where any display surface is curved as depicted in FIG. 10B. In this case, the transmission controller 011 is controlled in accordance with the distance of the object 001 from the probe 003 to any display surface on the image scan line 025. Consequently, the image quality of an image in any display surface set to be curved is effectively improved or uniformized. In this case, at each position that can taken by the probe, the distance of a living organism portion and the distances of portions other than the living organism (the matching material and the like), on a path between the probe and any display surface, are determined, and based on the resultant values, the intensity of the transmitted sound pressure is determined.

When the probe is moved depending on the roundness or unevenness of the object 001 as in the present embodiment, even front layer images can be easily created, and the output value difference within an image of any display surface can be corrected. As a result, the image quality on any display surface is restrained from being degraded, allowing easy-to-see images to be displayed.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-098270, filed on May 13, 2015, which is hereby incorporated by reference herein in its entirety.

What is claimed is:

1. An object information acquiring apparatus comprising:
a receiver including a plurality of elements each transmitting an acoustic wave, receiving an echo wave resulting from reflection of the acoustic wave by an object, and outputting an electric signal;
a transmission controller that controls an intensity of the acoustic wave transmitted from each of the plurality of elements;
a scanner that moves the receiver in a predetermined scanning region; and
an information processor that acquires characteristics information on an inside of the object using the electric signal,
wherein the transmission controller controls the intensity of the acoustic wave in accordance with a shape of the object and a position of the receiver in the predetermined scanning region.

2. The object information acquiring apparatus according to claim 1, wherein the transmission controller increases the intensity of the acoustic wave at a portion of the object where the object protrudes with respect to the receiver.

3. The object information acquiring apparatus according to claim 1, wherein the transmission controller reduces the intensity of the acoustic wave at a portion of the object where the object is depressed with respect to the receiver.

4. The object information acquiring apparatus according to claim 1, wherein the predetermined scanning region is a scanning plane shaped in a form of a flat surface, and
the transmission controller acquires a distance between the receiver and a surface of the object in a normal direction of the scanning plane based on a shape of the object, and controls the intensity of the acoustic wave according to the distance.

5. The object information acquiring apparatus according to claim 4, wherein the transmission controller increases the intensity of the acoustic wave proportionally with a decrease in the distance between the receiver and the surface of the object.

6. The object information acquiring apparatus according to claim 1, wherein the transmission controller applies a pulse that is set based on a voltage value and a pulse width to each of the plurality of elements so as to allow each of the plurality of elements to transmit the acoustic wave.

7. The object information acquiring apparatus according to claim 6, wherein the transmission controller increases the voltage value of the pulse to increase the intensity of the acoustic wave.

8. The object information acquiring apparatus according to claim 6, wherein the transmission controller increases the pulse width of the pulse to increase the intensity of the acoustic wave.

9. The object information acquiring apparatus according to claim 6, wherein the transmission controller increases a number of transmitted pulses of the pulse to increase the intensity of the acoustic wave.

10. The object information acquiring apparatus according to claim 6, wherein the transmission controller selects an element of the plurality of elements to which the pulse is applied to form a transmission aperture, and increases a number of elements included in the transmission aperture to increase the intensity of the acoustic wave.

11. The object information acquiring apparatus according to claim 1, wherein the transmission controller controls the intensity of the acoustic wave by changing a transmission focus position when the acoustic wave is transmitted from the plurality of elements.

12. The object information acquiring apparatus according to claim 1, wherein the transmission controller performs pre-scanning that is transmission and reception of the acoustic wave for acquiring a shape of the object before performing production imaging that is transmission and reception of the acoustic wave for acquiring the characteristics information.

13. The object information acquiring apparatus according to claim 1, further comprising a camera that acquires an image of the object,
wherein the transmission controller acquires the shape of the object using the image acquired by the camera.

14. The object information acquiring apparatus according to claim 1, further comprising a holder that holds the object,
wherein the transmission controller acquires the shape of the object based on information on the holder.

15. The object information acquiring apparatus according to claim 14, further comprising a memory that stores a transmission condition specified for each coordinate on the scanning region based on a shape of the holder and used by the transmission controller to control the intensity of the acoustic wave.

16. The object information acquiring apparatus according to claim 1, wherein the information processor corrects a gain to the electric signal in accordance with attenuation of the acoustic wave or the echo wave.
